(19) United States
(12) Patent Application Publication
(10) Pub. No.: US 2025/0389639 A1
(43) Pub. Date: Dec. 25, 2025

(54) APPARATUS FOR PRESENTING SMALL SAMPLES OF FIBROUS MATERIAL TO A MEASUREMENT DEVICE WHILE HARVESTING

(71) Applicant: RCI Engineering, LLC, Mayville, WI (US)

(72) Inventors: Randall J. Clark, Mayville, WI (US); Hunter M. Eilbes, Mayville, WI (US); Thomas J. Gehring, Mayville, WI (US); Alex J. Shute, Beaver Dam, WI (US); Larry O. Villwock, Mayville, WI (US); Ross D. Villwock, Mayville, WI (US); Aaron L. Weigel, Beaver Dam, WI (US)

(73) Assignee: RCI Engineering, LLC, Mayville, WI (US)

(21) Appl. No.: 19/243,267

(22) Filed: Jun. 19, 2025

Related U.S. Application Data
(60) Provisional application No. 63/662,598, filed on Jun. 21, 2024.

(51) Int. Cl. G01N 21/13; G01N 21/01; G01N 21/85; G01N 22/04

(52) U.S. Cl. CPC G01N 21/13; G01N 21/85; G01N 22/04; G01N 2021/0106; G01N 2021/8592

(57) ABSTRACT

A forage harvester from which chopped crop material is conveyed into a receiving chamber having a rotor and a testing device mounted in a lower portion of the receiving chamber. The rotor is formed with a plurality of tines that compress severed crop material at a consistent density past a sensor face of the testing device. The sensor face includes a near-infrared sensor or a microwave sensor, to analyze the crop material passing over the sensor face and stores the analysis in the data collection device. A spring-loaded resistance pan is mounted below the sensor face to help maintain density in the crop material compressed downwardly by the tips of the rotor tines. When the compression forces exerted by the rotor are greater than the biasing force of the resistance pan, the resistance pan yields to allow passage of the crop material into the upper hopper.

### CROSS-REFERENCE TO RELATED APPLICATIONS

This patent application claims domestic priority on co-pending U.S. Provisional Patent Application Ser. No. 63/662,598, filed on Jun. 21, 2024, the content of which is incorporated herein by reference.

### FIELD OF THE INVENTION

The present invention relates generally to testing crop material from small plots to determine moisture content and other parameters related to the quality of the crop material, and, more particularly, to a testing apparatus operable in conjunction with a harvesting machine while the harvesting machine is being operated in the field.

### BACKGROUND OF THE INVENTION

Measurement devices are well known in the agricultural industry to determine moisture content of the crop material and other pertinent parameters relating to the quality of the crop material being harvested, including sugar and protein content. Known measurement devices include a Near-Infrared (NIR) and microwave sensors. Traditional testing of crop material would involve the hand collection of a sample of the harvested crop material from a hopper into which the harvester crop material has been deposited. The operator would normally stop the operation of the harvesting machine before leaving the harvesting machine to hand-collect the sample of harvested crop material. The operator would then bag the hand-collected sample and place the bag in a storage location before returning to the harvesting machine and moving on to the next test plot. Later the hand-collected sample would be vacuum sealed in the bag, then refrigerated and subsequently shipped to a laboratory for analysis using an NIR or microwave sensor.

This traditional process of collecting data relating to the quality of the crop material being harvested would typically take about a week or two before the results were known about the crop at the time of harvesting. This process also resulted in inaccuracies and errors due to the handling of the samples, the changes in temperature of the samples and other processing problems. Testing crop material during harvesting operations is affected by variances in crop volume, as test plots will have disparaging yields, resulting in an uneven flow rate through the testing apparatus. This uneven flow rate provides inconsistent, non-reliable results for the purpose of measurement of crop parameters, thereby rendering known in-field testing of crop material with abundant inaccuracies.

Accordingly, it would be desirable to provide a crop material testing apparatus that can be used in the field at the time of harvesting the crop from the test plots in a manner that will provide reliable results and simple operation.

### SUMMARY OF THE INVENTION

It is an object of this invention to overcome the disadvantages of the prior art by providing a crop material testing apparatus that can be used in the field at the time of harvesting the crop.

It is another object of this invention to provide an apparatus that will test crop being harvested.

It is a feature of this invention that the apparatus includes a testing device that includes a sensor face that is exposed to the hopper of the harvester.

It is an advantage of this invention that the harvester includes a rotor having a plurality of tines operable upon rotation to move the harvested crop material downwardly across the sensor face of the testing device.

It is another advantage of this invention that the rotor maintains a consistent density in the crop material being passed along the sensor face of the testing device.

It is another feature of this invention that the testing device further includes a biased resistance pan pivotally mounted on the hopper below the sensor face.

It is still another advantage of this invention that the biased resistance pan helps in maintaining a consistent density in the harvested crop material passing across the sensor face.

It is still another feature of this invention that the tines of the rotor compress the harvested crop against the biased resistance pan.

It is yet another advantage of this invention that the force of compression on the crop is greater than the force of the spring biasing the resistance pan to allow the resistance pan to pivot and permit the crop to move further downwardly.

It is yet another feature of this invention that the rotor is cooperative with a stripper plate to clean harvested crop material from the tines.

It is a further feature of this invention that the harvester has an upper hopper and a lower hopper separated by a pair of doors movable between a closed position and an opened position.

It is still another object of this invention to provide the testing device for a forage harvester operable to sever crop from the field and to chop the severed crop into small pieces.

It is yet another object of this invention to provide a method of testing crop material for desired parameters while the crop is being harvested.

It is another feature of this invention to convey the severed crop material into a receiving chamber having a rotor mounted in a lower portion thereof.

It is still another feature of this invention that the rotor includes tines that compress the severed crop material along a sensor face of a testing device to determine desired parameters of the severed crop material.

It is another advantage of this invention that the method of testing maintains a consistent density of crop material presented to the sensor face irrespective of the volume of crop material conveyed into the receiving chamber.

It is yet another feature of this method to place a biased resistance pan below the sensor face and oriented generally perpendicular to the path of the severed crop material pushed downwardly by the rotor tines.

It is still another advantage of this invention that the biasing force of the resistance pan yields to the pressure of the compression force of the severed crop material from the rotor tines to allow passage of the crop material into an upper hopper.

It is a further feature of this invention that the upper hopper is supported on load cells to enable a weighing of the crop material accumulated in the upper hopper.

It is yet another advantage of this invention that the floor of the upper hopper is formed with a door that opens to access a lower hopper into which the accumulated crop material in the upper hopper can be discharged.

It is a further object of this invention to provide a testing device for a crop harvester to enable testing of the harvested crop material for desired parameters, the testing device being durable in construction and simple and effective in use.

These and other objects, features and advantages are accomplished according to the instant invention by providing an agricultural harvester, such as a forage harvester, from which chopped crop material is conveyed into a receiving chamber having a rotor and a testing device mounted in a lower portion of the receiving chamber. The rotor is formed with a plurality of tines that compress severed crop material at a consistent density past a sensor face of the testing device. The sensor face includes a near-infrared sensor or a microwave sensor, to analyze the crop material passing over the sensor face and stores the analysis in the data collection device. A spring-loaded resistance pan is mounted below the sensor face to help maintain density in the crop material compressed downwardly by the tips of the rotor tines. When the compression forces exerted by the rotor are greater than the biasing force of the resistance pan, the resistance pan yields to allow passage of the crop material into the upper hopper.

### BRIEF DESCRIPTION OF THE DRA WINGS

The advantages of this invention will become apparent upon consideration of the following detailed disclosure of the invention, especially when taken in conjunction with the accompanying drawings wherein:

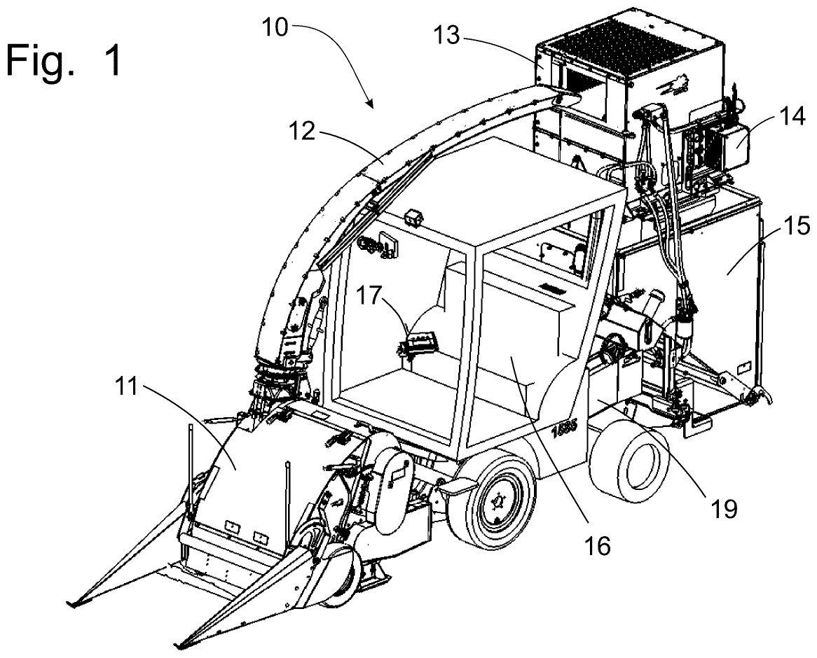

FIG. 1 is a left front perspective view of a research plot sample presentation unit incorporating the principles of the instant invention;

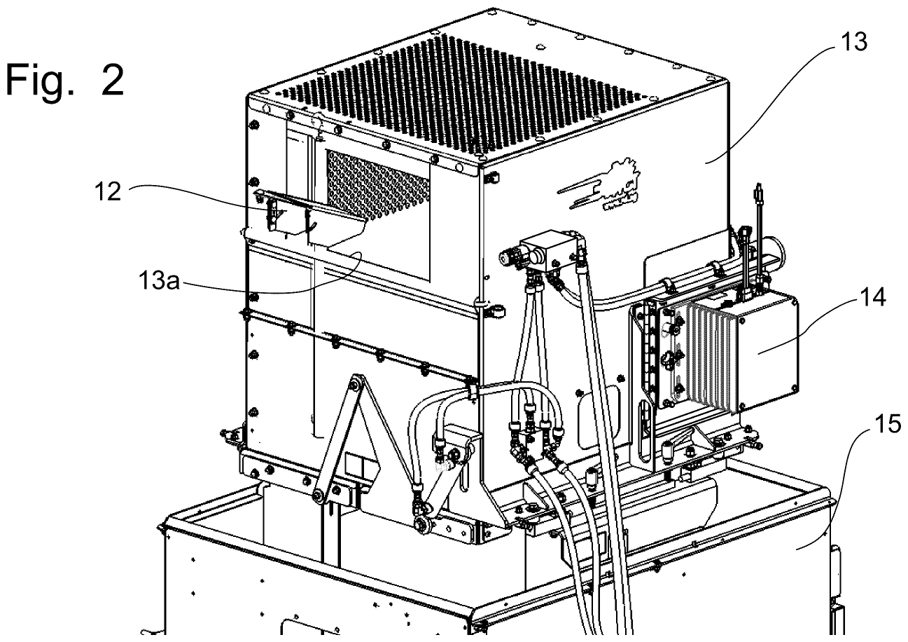

FIG. 2 is an enlarged left, front perspective view of the upper hopper receiving the harvested crop material from the crop harvester for testing;

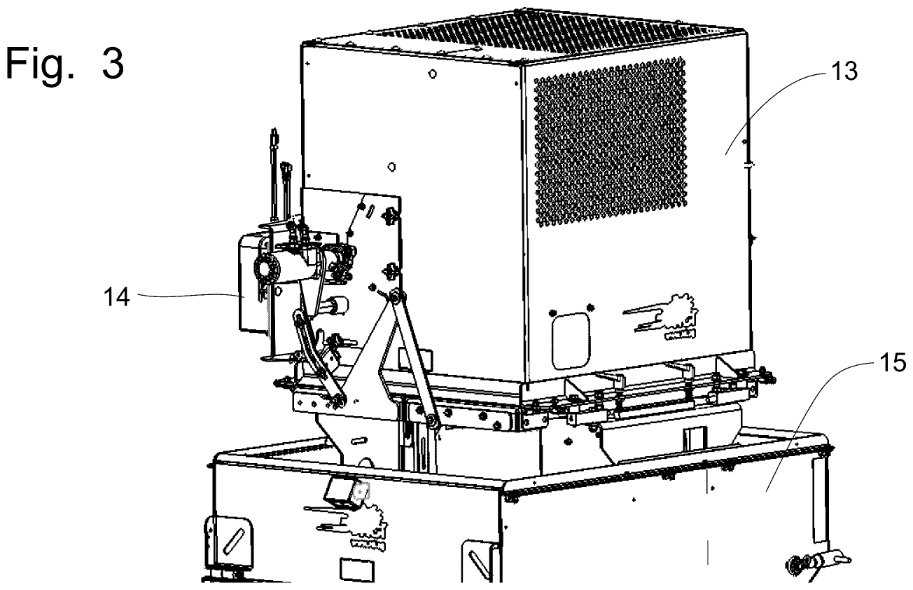

FIG. 3 is a right, rear perspective view of the upper hopper shown in FIG. 2;

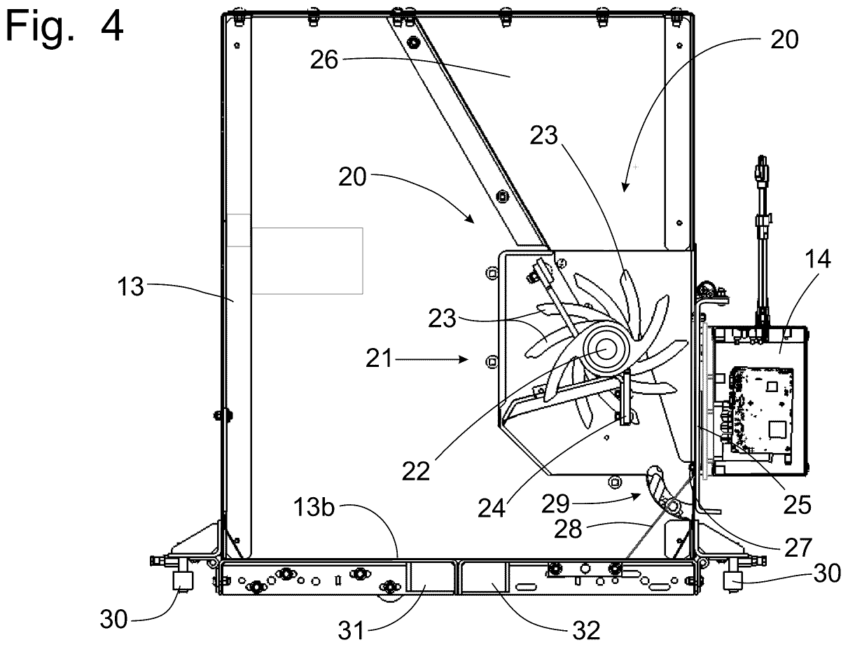

FIG. 4 is a front elevational view of the upper hopper with the front wall removed to provide clarity in a depiction of the sample presentation apparatus within the upper hopper;

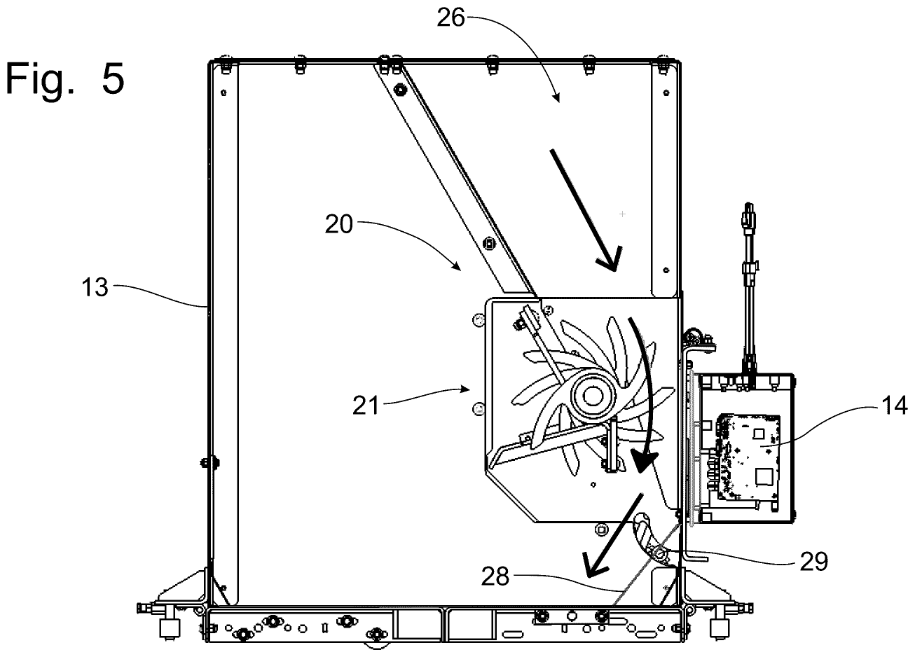

FIG. 5 is a front elevational cross-sectional view as shown in FIG. 4, but showing arrows indicating the direction of flow of crop material through the sample presentation apparatus;

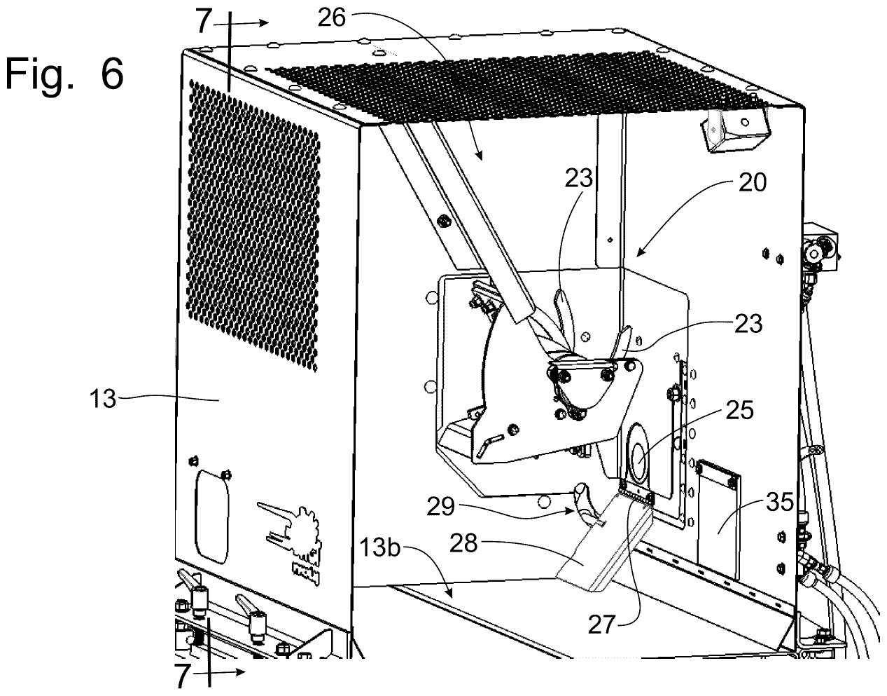

FIG. 6 is a right, front perspective cross-sectional view with the front wall of the upper hopper removed for purposes of clarity for depicting the sample presentation apparatus;

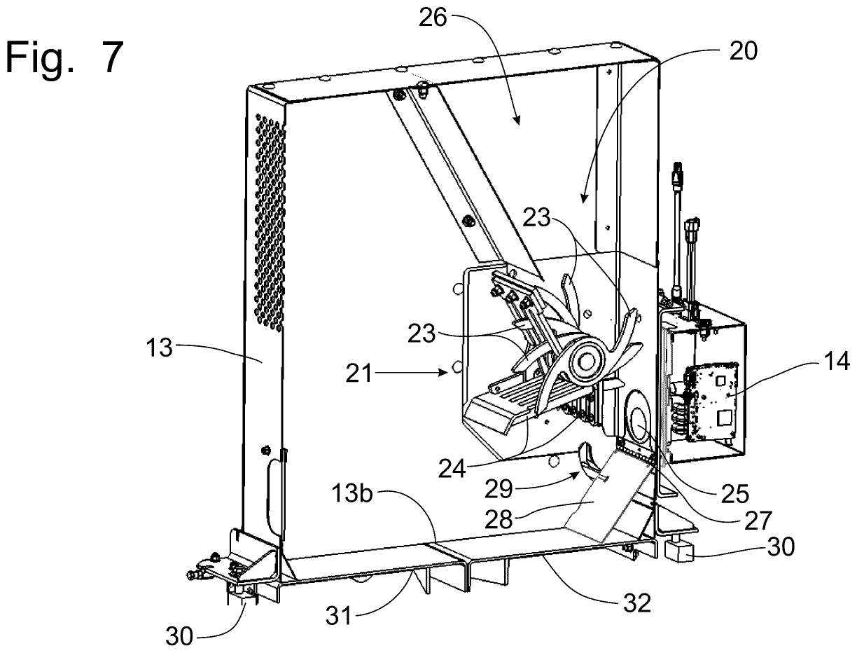

FIG. 7 is a right, front perspective cross-sectional view corresponding to lines 7-7 of FIG. 6 to provide further detail of the sample presentation apparatus;

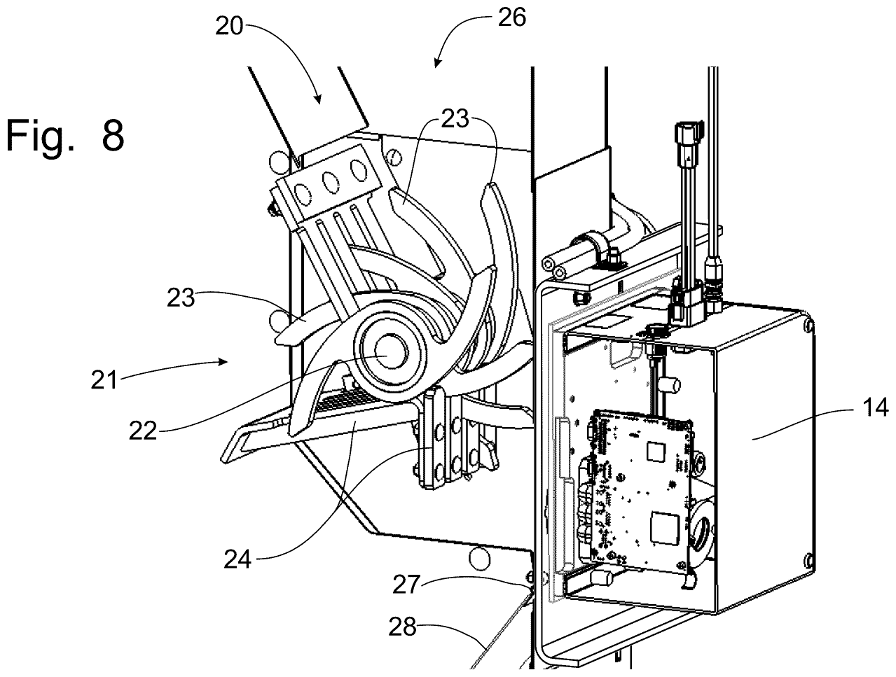

FIG. 8 is an enlarged left, front perspective view of the rotor assembly portion of the sample presentation apparatus; and

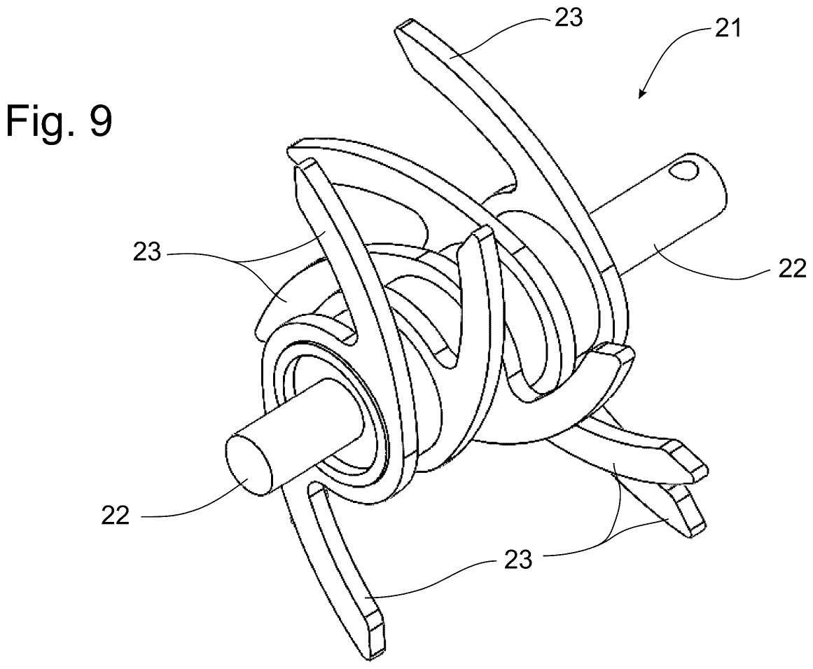

FIG. 9 is a perspective view of the rotor forming a part of the sample presentation apparatus.

### DETAILED DESCRIPTION OF THE PREFERRED EMBODIMENT

Referring to the drawings, a preferred embodiment of an in-field sample presentation unit for measurement of parameters of harvested crop material from a test plot, incorporating the principles of the instant invention, can best be seen. The sample presentation unit 10 includes a forage harvester 11 sized for the harvesting of samples of crop material from a research test plot for the crop material and having a header 11a that collects severed crop material from the test plot, chops the severed crop material into discrete pieces and blows the chopped crop material through a conventional spout 12 into an upper hopper 13 for delivery through an opening 13a to the sample presentation apparatus 20 supported within the upper hopper 13. A conventional power unit 19 provides operational power for the forage harvester 11, header 11a and the sample presentation apparatus 20. The testing device 14 is mounted on the upper hopper for operation as will be described in greater detail below. A lower hopper 15 is positioned below the upper hopper 13 to received harvested crop material therefrom. A data collection device 17, such as a microprocessor, is preferably supported on the operator station 16 for convenient access by the operator.

Referring now to FIGS. 4-8, the placement of the sample presentation apparatus 20 in the upper hopper 13 locates a receiving chamber 26 at a position to receive a portion of the stream of crop material discharged through the spout 12. The flow of crop material, as depicted in FIG. 5, is funneled down toward a rotor 21, rotatable about a horizontal axis 22 to engage the crop material by curved tines 23 that convey the crop material downwardly past the testing device 14. The rotor 21 is formed with a plurality of tines 23 spaced circumferentially around the rotor axle 22. As the tines rotate into the crop material, the tines 23 pull the crop material downwardly such that the tip of the tines push the crop material past the sensor face 25 toward the resistance pan 28.

The resistance pan 28 is positioned below the face 25 of the testing device 14 to reduce the opening at the bottom of the receiving chamber 26 until sufficient pressure is created by the crop material being analyzed by the testing device 14, as will be described in greater detail below. The resistance pan 28 is spring loaded by the spring mechanism 29 so as to be biased into a raised position and pivotable about the hinge 27 mounting the resistance pan 28 to the wall of the upper hopper 13. As the tines 23 continue to push crop material past the sensor face 25 and into engagement with the resistance pan 28, pressure from the pushing of the crop material against the resistance pan 28 increases until the pressure overcomes the spring bias exerted by the spring mechanism 29 and pivots the resistance pan 28 about the hinge 27 to allow the accumulated crop material to move to the bottom of the upper hopper 13.

The rotor 21 is rotated at relatively slow speeds and only advances the crop material at the sensor face 25 when the crop material is delivered to the inlet side of the rotor 21 and then compressed downwardly by the tips of the tines 23. While the rotor 21 continues to rotate continuously during operation of the sample presentation unit 10, the crop material will not advance past the sensor face 25 until additional crop material is received within the receiving chamber 26 and compressed downwardly by the tines 23. The details of the rotor 21 are depicted in FIG. 9. The tines 23 are mounted on the rotor axle 22 in an opposing manner and are positioned in alignment with slots in stripper plates 24 to remove crop material from the tines 23 and prevent the crop material from wrapping around the rotor 21. A door 35 is provided in the upright wall of the upper hopper 13 near the floor 13b to provide access to the crop material accumulated at the bottom of the upper hopper 13 in order to acquire a hand sample should one be desired.

In operation, the forage harvester 11 severs crop material from the test plot, chops the crop material and blows the chopped crop material rearwardly via the spout 12 through the opening 13a in the upper hopper 13 to be received within the receiving chamber 26. The received crop material flows downwardly toward the rotor 21 where the tines 23 press the crop material past the sensor face 25 and into engagement with the resistance pan 28. The testing device 14, which is preferably a near-infrared sensor or a microwave sensor, analyzes the crop material passing over the sensor face 25 and stores the analysis in the data collection device 17. The stripper plates 24 clean the crop material from the tines 23 as the tines 23 rotate back into engagement with the crop material.

The resistance pan 28 is spring-loaded into an upright position to restrict the exit of the upper hopper 13 and allow the crop material to be compressed by the tines 23 onto the resistance pan 28 with the sensor face 25 being located above the resistance pan 28. In the event of a reduction in crop material being placed into the receiving chamber 26, the crop material remains compressed against the resistance pan 28 in front of the sensor face 25 until more crop material is placed into the receiving chamber 26 and compressed toward the resistance pan 25 by the tines 23. Accordingly, the testing device 14 sees a consistent amount of crop material before the sensor face for accurate readings and analysis.

The bias exerted by the spring mechanism 29 keeps the resistance pan 28 in an upright orientation generally perpendicular to the flow of crop material as the crop material is being compressed past the sensor face 25 against the upright resistance pan 28. With the continued compaction of crop material by the tines 23, the pressure on the resistance pan 28 increases to the point that the spring bias exerted by the spring mechanism 29 yields to the pressure and allows the resistance pan 28 to rotate downwardly about the hinge 27 and allow crop material to move toward the bottom of the upper hopper 13.

Once the test plot has been harvested and the crop material is accumulated in the bottom of the upper hopper 13, the weight of the crop material harvested from the test plot is recorded from load cells 30 that support the upper hopper 13. The doors 31, 32 forming the floor 13b of the upper hopper 13 are then rotated about the respective pivots 33 to drop the accumulated crop material into the lower hopper 15. With the opening of the doors 31, 32, the spring mechanism 29 is released to allow the resistance pan 28 to drop vertically and clear the sensor face 25 from crop material. The doors 31, 32 are then returned to their closed positions, and the resistance pan 28 reset, for harvesting the next test plot. Upon completion of the harvesting and analysis of the crop material from the subsequent test plot, the doors 31, 32 are again opened to allow the accumulated crop material to drop into the lower hopper 15. When the lower hopper 15 is filled with accumulated crop material from the multiple test plots, the sample presentation unit 10 is returned to home to empty the lower hopper 15.

One skilled in the art will recognize that the crop material can be processed as fast or as slow as the crop material is harvested and placed into the receiving chamber 26. While interruptions of the crop flow may occur during the harvesting operations, the interruption of crop flow will not impact the operation of the test device 14 because the interruption of the flow of crop material will not clear the sensor face 25 of crop material compacted against the resistance pan 28 because the tips of the tines 23 of the rotor 21 are spaced above the sensor face 25 and do not act to clear material off of the sensor face 25.

The design of the rotor 21 is such that the tines will compress and move crop material as crop material is made available to the rotor 21, but will not push crop material any further than the distal end of the tines 23 as the tines 23 are rotated. The back side of the tines 23 can accept larger volumes of crop material and be sufficiently aggressive to force the crop material to the sensor face 25 in varying moisture conditions, and in varying volumes. This rotor design ensures a quality presentation of crop material to the sensor face 25 in a consistent density for accurate readings and consistent analysis by the test device 14.

It will be understood that changes in the details, materials, steps and arrangements of parts, which have been described and illustrated to explain the nature of the invention will occur to and may be made by those skilled in the art upon a reading of this disclosure within the principles of the scope of the invention. The foregoing description illustrates the preferred embodiment of the invention; however, concepts, as based upon the description may be employed in other embodiments without departing from the scope of the invention. Accordingly, the following claims are intended to protect the invention broadly, as well as in the specific form shown.

Having thus described the invention, what is claimed is:

1. An apparatus for testing crop material received from a harvesting machine, comprising:
a receiving chamber operable to receive crop material from said harvesting machine;
a rotor positioned at a discharge opening of said receiving chamber, said rotor including a plurality of tines operable with the rotation of said rotor about an axis of rotation to move the crop material downwardly; and
a testing device having a sensor face exposed to said rotor, said tines being operable to push the crop material across said sensor face from the supply of the crop material being received by said receiving chamber, said rotor maintaining a consistent density of the crop material in front of said sensor face irrespective of the flow of the crop material received by said receiving chamber.

2. The apparatus of claim 1 wherein said receiving chamber is mounted on an upper hopper, said apparatus further comprising:
a biased resistance pan pivotally connected to said upper hopper and being positioned below said sensor face, said resistance pan being operable to assist in maintaining said crop material at a consistent density in front of said sensor face.

3. The apparatus of claim 2 wherein said tines push crop material downwardly across said sensor face to compress said crop material against said resistance pan, the compression of said crop material greater than the bias of the resistance pan causing the bias to yield and allow the resistance pan to pivot so as to allow the crop material to move further downwardly into said upper hopper.

4. The apparatus of claim 3 further comprising:
a stripper plate in communication with said tines as said tines rotate about said axis of rotation to clean said crop material from said tines.

5. The apparatus of claim 3 further comprising:
said upper hopper having a floor formed by at least one pivoted door movable between a closed position and an opened position, said closed position permitting an accumulation of crop material thereon, said at least one door being operatively connected to said resistance pan to release the bias thereon when said at least one door moves to said opened position, the return of said at least one door to said closed position causing the bias for said resistance pan to be reset.

6. The apparatus of claim 5 wherein said upper hopper includes said receiving chamber and rests on a lower hopper that receives crop material from said hopper when said at least one door is moved to said opened position.

7. The apparatus of claim 3 wherein said hopper includes a receiving chamber funnels the crop material received therein toward engagement with said rotor.

8. The apparatus of claim 3 wherein said harvesting machine is a forage harvester operable to sever crop from the ground, chop the severed crop material into pieces and convey said pieces of crop material into said receiving chamber.

9. The apparatus of claim 1 wherein said testing device is a near-infrared sensor or a microwave sensor.

10. A harvesting machine operable to sever crop material from the field for further processing, comprising:
a receiving chamber supported on a hopper and being operable to receive severed crop material from said harvesting machine;
a rotor positioned at a discharge opening of said receiving chamber, said rotor including a plurality of tines operable with the rotation of said rotor about an axis of rotation to move the severed crop material downwardly through said discharge opening; and
a testing device having a sensor face exposed to said rotor, said tines being operable to push the severed crop material across said sensor face as the severed crop material is received by said rotor, said rotor maintaining a consistent density of the crop material in front of said sensor face irrespective of the flow of the severed crop material received by said receiving chamber.

11. The harvesting machine of claim 10 further comprising:
a spring-loaded resistance pan pivotally connected to said hopper and being positioned below said sensor face, said resistance pan being operable to assist in maintaining said severed crop material at a consistent density in front of said sensor face.

12. The harvesting machine of claim 11 wherein said tines push severed crop material downwardly across said sensor face to compress said severed crop material against said resistance pan, the compression of said severed crop material greater than the bias of the spring-loaded resistance pan causing the bias to yield and allow the resistance pan to pivot so as to allow the severed crop material to move further downwardly into said hopper.

13. The harvesting machine of claim 10 further comprising:
a stripper plate in communication with said tines as said tines rotate about said axis of rotation to clean said severed crop material from said tines.

14. The harvesting machine of claim 11 further comprising:
said hopper having a floor formed by at least one pivoted door movable between a closed position and an opened position, said closed position permitting an accumulation of severed crop material thereon, said at least one door being operatively connected to said resistance pan to release the bias thereon when said at least one door moves to said opened position, the return of said at least one door to said closed position causing the bias for said resistance pan to be reset.

15. The harvesting machine of claim 14 wherein the opening of said at least one pivoted door allows movement of the severed crop material in said hopper to discharge into a lower hopper.

16. The harvesting machine of claim 10 wherein said harvesting machine is a forage harvester operable to sever crop from the ground, chop the severed crop material into pieces and convey said pieces of crop material into said receiving chamber.

17. The harvesting machine of claim 10 wherein said testing device is a near-infrared sensor or a microwave sensor.

18. A method of testing crop material for desired parameters by a testing device, comprising the steps of:
harvesting crop material from a plot of ground by severing said crop material from the ground;
conveying the severed crop material into a receiving chamber having a rotor mounted at a bottom portion of said receiving chamber for rotation about an axis of rotation, said rotor including a plurality of spaced-apart tines pushing said crop material downwardly;
mounting said testing device with a sensor face positioned below said rotor such that said tines do not push crop material away from said sensor face;
maintaining a consistent density of crop material presented to said sensor face, irrespective of the volume of crop material being conveyed into said receiving chamber; and
operating said testing device through said sensor face to detect the desired parameters of said crop material.

19. The method of claim 18 further comprising the step of:
placing a resistance pan below said sensor face, said resistance pan being biased into a first position substantially perpendicular to the path of crop material pushed downwardly by said rotor, said resistance pan yielding to pressure from a compression of crop material against said resistance pan asserted by said rotor.

20. The method of claim 19 wherein said receiving chamber is positioned within an upper hopper, said method further comprising the steps of:
weighing accumulate crop material through a support of said upper hopper on load cells; and
supporting said upper hopper on a lower hopper, said upper hopper having a pair of doors forming a floor of said upper hopper so that a selected opening of said doors would discharge accumulated crop material into said lower hopper for transport thereof.
